(19) United States
(12) Patent Application Publication
(10) Pub. No.: US 2006/0252096 A1
(43) Pub. Date: Nov. 9, 2006

(54) Single chain antibody with cleavable linker

(75) Inventors: Dongxing Zha, Lebanon, NH (US); Byung-Kwon Choi, Norwich, VT (US); Tillman U. Gerngross, Hanover, NH (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: GlycoFi, Inc., Lebanon, NH (US)

(21) Appl. No.: 11/411,436

(22) Filed: Apr. 25, 2006

Related U.S. Application Data
(60) Provisional application No. 60/675,218, filed on Apr. 26, 2005.

(51) Int. Cl. G01N 33/53; C07H 21/04; C12P 21/06; C12N 1/18; C07K 16/44; C12N 15/74

(52) U.S. Cl. 435/7.1; 435/69.1; 435/254.1; 435/254.2; 435/483; 530/388.15; 536/23.53

(57) ABSTRACT

Disclosed are antibodies and methods for making antibodies with desired glycosylation and efficient production. Host cells transformed with a nucleic acid encoding a fusion protein comprising a signal sequence, light and heavy immunoglobulin chains, each comprising a variable region and a constant region and separated by a spacer peptide comprising at least one proteolytic cleavage site are cultured to express the nucleic acids and are cleaved by appropriate proteases to produce antibodies.

### CROSS-REFERENCE TO RELATED APPLICATION

The present application is a nonprovisional of 60/675,218 filed Apr. 26, 2005, incorporated by reference in its entirety for all purposes.

### BACKGROUND OF THE INVENTION

Although mammalian cellular systems have been successfully used to produce antibodies for therapeutic uses, they are expensive to develop and there is presently insufficient manufacturing capacity to meet anticipated future needs for antibodies. Cellular systems for expression of proteins from lower eukaryotes and bacteria are cheaper and simpler to operate but are associated with other difficulties in producing antibodies. Most previous work in these cell types has been confined to expression of antibody fragments rather than intact antibodies (see, e.g., Better et al., Science 240, 1041-1043 (1988)) due to poor folding and/or yield of intact antibodies in such systems. However, antibody fragments are only useful in situations where binding of an antibody is sufficient for therapeutic activity; in other words, when effector function is not needed.

Even when effector function is not required, antibodies produced by prokaryotes and lower eukaryotes are often less useful than those from mammalian cells due to lack of appropriate glycosylation. Different organisms produce different glycosylation enzymes (e.g. glycosyltransferases and glycosidases), and have different substrates (nucleotide sugars) available, so that the glycosylation patterns as well as composition of the individual oligosaccharides, even of the same protein, are different depending on the host system in which the particular protein is being expressed. Bacteria typically do not glycosylate proteins, and if so only in a very unspecific manner (Moens and Vanderleyden, Arch Microbiol. 168(3):169-175 (1997)). Lower eukaryotes such as filamentous fungi and yeast add primarily mannose and mannosylphosphate sugars. The resulting glycan is known as a “high-mannose” type glycan or a mannan. Plant cells and insect cells (such as Sf9 cells) glycosylate proteins in yet another way. By contrast, in higher eukaryotes such as humans, the nascent oligosaccharide side chain may be trimmed to remove several mannose residues and elongated with additional sugar residues that typically are not found in the N-glycans of lower eukaryotes (Coloma et al., 2000, Mol. Immunol. 37: 1081-1090; Raju et al., 2000, Glycobiology, 10: 477-486; Weikert, et al. Nature Biotechnology, 1999, 17, 1116-1121; Malissard, et al. Biochemical and Biophysical Research Communications, 2000, 267, 169-173). Lack of appropriate glycosylation in antibodies produced by bacteria or lower eukaryotes can adversely affect immunogenicity, pharmacokinetic properties, trafficking, and efficacy of therapeutic proteins.

One form of antibody fragment that has been expressed in bacteria and lower eukaryotes is known as a single-chain antibody (see e.g., U.S. Pat. No. 4,946,778 and U.S. Pat. No. 5,132,405). Such molecules include a light chain variable region separated by a spacer peptide from a heavy chain variable region, but typically lack some and usually all of the constant regions. The single-chain antibody form is particularly suitable for screening large numbers of antibodies for a desired binding specificity, particularly by phage display (see, e.g., McCafferty et al., Nature 348, 552, 554 (1990)). In such screens, the smaller size of single-chain antibodies relative to intact antibodies is advantageous for obtaining display without impairing viability of the phage, and the lack of constant region is irrelevant to binding specificity. The small size of single-chain antibodies lacking constant regions has also been proposed as having advantages for therapeutic purposes in applications of antibodies not requiring effector functions (Cochet et al., Cancer Detect. Prev., 23, 506-510 (1999); McCall et al., Mol. Immunol., 36, 433-445 (1999); Pavlinkova et al., J. Nuclear Med., 40, 1536-1546 (1999)). The small size has been proposed to lead to improved penetration of a target tissue. The small size has also been reported to be advantageous in reducing incorrect folding because of the decreased number of potential antibody conformations (Jaeger et al., FEBS Letters, 462, 307-312 (1999)).

### BRIEF SUMMARY OF THE INVENTION

The invention provides methods of producing an antibody. The methods comprise culturing a fungal cell transformed with a nucleic acid encoding a fusion protein comprising in order from N-terminus to C-terminus: (a) a signal sequence, (b) a first immunoglobulin chain comprising a variable region and a constant region, (c) a spacer peptide comprising a proteolytic cleavage site cleavable by a protease which is a separate molecule from the fusion protein, and (d) a second immunoglobulin chain comprising a variable region and a constant region; wherein the first immunoglobulin chain is a light chain and the second immunoglobulin chain is a heavy chain, or vice versa; the fusion protein is free of a second signal sequence between the spacer peptide and the second immunoglobulin chain; and the spacer peptide lacks a self-processing cleavage site. The fusion protein is expressed, cleaved at the C-terminal end of the signal sequence to remove the signal sequence, and cleaved at the proteolytic site in the spacer peptide by the protease. A antibody comprising a pair of intermolecularly associated immunoglobulin heavy and light chains is produced.

Preferably the antibody is a tetrameric antibody comprising two pairs of the intermolecularly associated immunoglobulin heavy and light chains. In some methods, the first immunoglobulin chain is a light chain and the second immunoglobulin chain is a heavy chain. In other methods, the first immunoglobulin chain is a heavy chain and the second immunoglobulin chain is a light chain.

In some methods, the light and heavy chains of the fusion protein associate with each other by intramolecular bonding, and two copies of the fusion protein associate with each other by intermolecular bonding of their respective heavy chain constant regions before cleavage at the proteolytic site occurs. In some methods, cleavage at the proteolytic site is followed by intermolecular association of the immunoglobulin heavy and light chains to form the pair of intermolecularly associated heavy and light chains, and intermolecular association between two pairs of the intermolecularly associated heavy and light chains to form the tetrameric antibody. In some methods, the spacer peptide comprises first and second proteolytic cleavage sites cleavable by first and second proteases, both proteases being separate molecules from the fusion protein, wherein the first and second proteolytic cleavage sites are separated by a peptide linker, and cleavage of the proteolytic cleavage sites by the first and second proteases removes the peptide linker from the fusion protein. In some methods, the first and second protease are the same protease.

In some methods, the cleavage of the first and second proteolytic sites occurs in the cell. In some methods, the cell secretes the antibody. In some methods, the cell is transformed with a nucleic acid encoding the protease that cleaves the first and second proteolytic sites. In some methods, the nucleic acid encodes a second fusion protein comprising a second signal sequence fused to the protease, wherein the second signal sequence causes uptake of the protease into the endoplasmic reticulum. In some methods, the fusion protein is secreted from the cell without the signal sequence, and the method further comprises treating the secreted fusion protein with the protease, which cleaves the proteolytic site in the spacer peptide.

Some methods further comprise recovering the antibody from the cell or from media in which the cell is cultured. Some methods further comprise purifying the antibody to essential homogeneity. Some methods further comprise combining the antibody with a pharmaceutical carrier in a pharmaceutical composition. Some methods further comprise introducing the nucleic acid encoding the fusion protein into the cell.

In some methods, the cell is a filamentous fungus cell. In some methods, the cell is a yeast cell. Preferred cells are from strains selected from the group consisting of cells from Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans Aspergillus nidulans, Aspergillus niger, Aspergillus oryzae, Trichoderma reesei, Chrysosporium lucknowense, Fusarium sp., Fusarium gramineum, Fusarium venenatum and Neurospora crassa.

In some methods, the proteolytic cleavage sites are Kex2p sites. Optionally, the proteolytic cleavage sites have the amino acid sequence XXKR, where X is any amino acid. Optionally, the proteolytic cleavage sites have the amino acid sequence XXKR, where X is any hydrophobic or hydrophilic amino acid. Optionally, the proteolytic cleavage sites have the amino acid sequence RHKR. Optionally, the spacer peptide has an N-terminal proteolytic cleavage site having the amino acid sequence LVKR and a C-terminal proteolytic cleavage site having the amino acid sequence RLVKR. Optionally, the antibody lacks all residues of the spacer peptide.

In some methods, the tetrameric antibody has an effector function. Optionally, the effector function is complement fixation or antibody dependent cellular toxicity.

In some methods, the immunoglobulin light chain and heavy chain are humanized immunoglobulin light and heavy chains.

In some methods, the antibody is produced at a yield of at least 50 mg/liter of culture medium. In some methods, the antibody is glycosylated at least at position Asn297.

In some methods, the heavy chain constant region comprises CH1, hinge, CH2, and CH3 regions. In some methods, the heavy chain constant region further comprises as CH4 region.

Some methods further comprise purifying the antibody and incorporating the antibody into a diagnostic kit.

In some methods, the fusion protein lacks peptide segments from a host protein between the signal sequence and the first immunoglobulin chain or between the peptide spacer and the second immunoglobulin chain.

The invention further provides a nucleic acid encoding a fusion protein comprising in order from N-terminus to C-terminus: (a) a signal sequence, (b) a first immunoglobulin chain comprising a variable region and a constant region, (c) a spacer peptide comprising a proteolytic cleavage site cleavable by a protease which is a separate molecule from the fusion protein, and (d) a second immunoglobulin chain comprising a variable region and a constant region; wherein the first immunoglobulin chain is a light chain and the second immunoglobulin chain is a heavy chain, or vice versa; the fusion protein is free of a second signal sequence between the spacer peptide and the second immunoglobulin chain; and the spacer peptide lacks a self-processing cleavage site. The invention also provides a vector comprising such a nucleic acid operably linked to a regulatory sequence, and a cell transformed with such a nucleic acid.

The invention further provides an antibody composition comprising a plurality of molecules of an antibody produced by the above methods in which each of the plurality has a glycoform, and the predominant glycoform is complex and lacking fucose. Optionally, the predominant glycan structure is present at a level that is at least about 10-25 mole percent more than the next predominant glycan structure of the antibody composition.

The invention further provides a monoclonal antibody produced by the above methods. Optionally, the monoclonal antibody specifically binds to EGFR, CD20, CD33, or TNF-alpha.

The invention further provides methods of producing an antibody. Such a method comprises culturing a cell transformed with a nucleic acid encoding a fusion protein comprising a signal sequence, an immunoglobulin light chain comprising a variable region and a constant region, a spacer peptide comprising first and second proteolytic cleavage sites cleavable by first and second proteases, which can be the same or different, both of which are separate molecules from the fusion protein, and an immunoglobulin heavy chain comprising a variable region and a constant region, wherein the spacer peptide is free of a self-cleavable proteolytic site, wherein the fusion protein is expressed, cleaved at the C-terminal end of the signal sequence to remove the signal sequence, and cleaved by the first and second proteases at the first and second proteolytic sites in the spacer peptide, and an antibody comprising a pair of intermolecularly associated immunoglobulin heavy and light chains is produced.

In some methods, the antibody is a tetrameric antibody comprising two pairs of the intermolecularly associated immunoglobulin heavy and light chains. In some methods, the first immunoglobulin chain is a light chain and the second immunoglobulin chain is a heavy chain. In other methods, the first immunoglobulin chain is a heavy chain and the second immunoglobulin chain is a light chain.

In some methods, the light and heavy chains of the fusion protein associate with each other by intramolecular bonding, and two copies of the fusion protein associate with each other by intermolecular bonding of their respective heavy chain constant regions before cleavage at the proteolytic site occurs. In some methods, cleavage at the proteolytic site is followed by intermolecular association of the immunoglobulin heavy and light chains to form the pair of intermolecularly associated heavy and light chains, and intermolecular association between two pairs of the intermolecularly associated heavy and light chains to form the tetrameric antibody.

In some methods, the spacer peptide comprises first and second proteolytic cleavage sites cleavable by first and second proteases, both proteases being separate molecules from the fusion protein, wherein the first and second proteolytic cleavage sites are separated by a peptide linker, and cleavage of the proteolytic cleavage sites by the first and second proteases removes the peptide linker from the fusion protein. In some methods, the first and second protease are the same protease.

In some methods, the cleavage of the first and second proteolytic sites occurs in the cell. In some methods, the cell secretes the antibody. In some methods, the cell is transformed with a nucleic acid encoding the protease that cleaves the first and second proteolytic sites.

In some methods, the nucleic acid encodes a second fusion protein comprising a second signal sequence fused to the protease, wherein the second signal sequence causes uptake of the protease into the endoplasmic reticulum. In some methods, the fusion protein is secreted from the cell without the signal sequence, and the method further comprises treating the secreted fusion protein with the protease, which cleaves the proteolytic site in the spacer peptide. Some methods further comprise recovering the antibody from the cell or from media in which the cell is cultured. Some methods further comprise purifying the antibody to essential homogeneity.

Some methods further comprise combining the antibody with a pharmaceutical carrier in a pharmaceutical composition.

Some methods further comprise introducing the nucleic acid encoding the fusion protein into the cell.

In some methods, the cell is a filamentous fungus cell. In some methods, the cell is a yeast cell. Preferred cells are selected from the group consisting of cells from Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichiapyperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans Aspergillus nidulans, Aspergillus niger, Aspergillus oryzae, Trichoderma reesei, Chrysosporium lucknowense, Fusarium sp., Fusarium gramineum, Fusarium venenatum and Neurospora crassa.

In some methods, the proteolytic cleavage sites are Kex2p sites. Optionally, the proteolytic cleavage sites have the amino acid sequence XXKR, where X is any amino acid. Optionally, the proteolytic cleavage sites have the amino acid sequence XXKR, where X is any hydrophobic or hydrophilic amino acid. Optionally, the proteolytic cleavage sites have the amino acid sequence RHKR. Optionally, the spacer peptide has an N-terminal proteolytic cleavage site having the amino acid sequence LVKR and a C-terminal proteolytic cleavage site having the amino acid sequence RLVKR. Optionally, the antibody lacks all residues of the spacer peptide.

In some methods, the tetrameric antibody has an effector function. Optionally, the effector function is complement fixation or antibody dependent cellular toxicity.

In some methods, the immunoglobulin light chain and heavy chain are humanized immunoglobulin light and heavy chains.

In some methods, the antibody is produced at a yield of at least 50 mg/liter of culture medium. In some methods, the antibody is glycosylated at least at position Asn297.

In some methods, the heavy chain constant region comprises CH1, hinge, CH2, and CH3 regions. In some methods, the heavy chain constant region further comprises as CH4 region.

Some methods further comprise purifying the antibody and incorporating the antibody into a diagnostic kit.

In some methods, the fusion protein lacks peptide segments from a host protein between the signal sequence and the first immunoglobulin chain or between the peptide spacer and the second immunoglobulin chain.

### BRIEF DESCRIPTION OF THE DRAWINGS

FIG. 1 shows heavy and light chains expressed as a single transcriptional and translational unit from a single set of transcriptional and translational regulatory sequences.

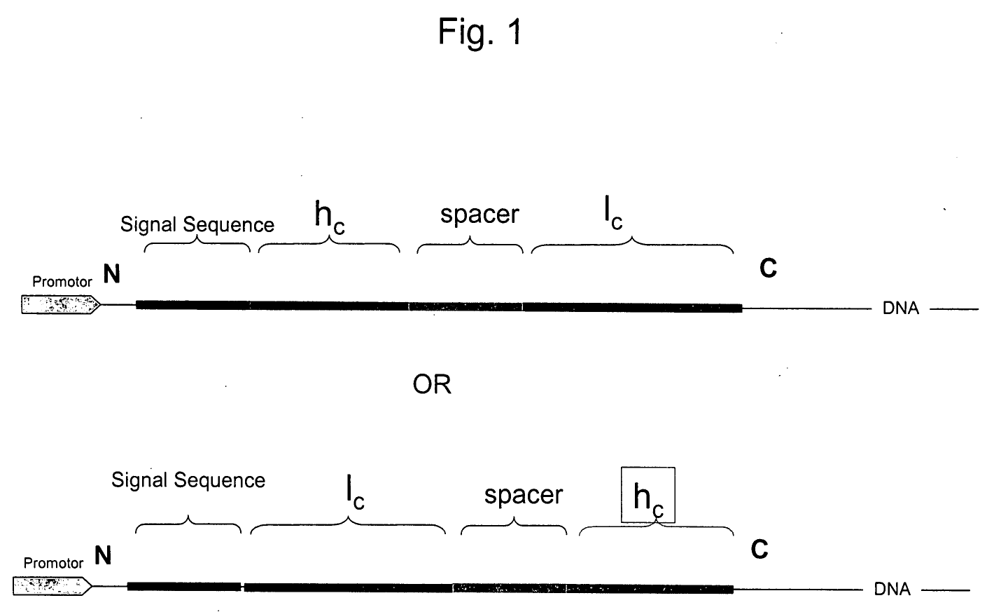

FIG. 2 shows an assembled antibody with peptide spacers

FIG. 3 shows peptide spacers cleaved in vitro or in vivo leaving fully assembled antibody.

FIG. 4 shows a construct used for expressing a single chain antibody.

FIG. 5 shows a gel confirming expression of a tetrameric antibody.

FIG. 6 shows binding of antibody dimer to FcγRIIIA-LV.

FIG. 7 shows binding of antibody to its antigen.

### DEFINITIONS

The basic antibody structural unit is known to comprise a tetramer of subunits. Each tetramer has two identical pairs of polypeptide chains, each pair having one “light” chain (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. The light and heavy chains are subdivided into variable regions and constant regions (See generally, Fundamental Immunology (Paul, W., ed., 2nd ed. Raven Press, N.Y., 1989), Ch. 7 (incorporated by reference in its entirety for all purposes).

The variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The heavy chain constant region is subdivided into CH1, hinge, CH2, CH3 and in some cases, CH4 regions. The assignment of amino acids to each domain and numbering of amino acids is in accordance with the definitions of Kabat, Sequences of Proteins of Immunological Interest (National Institutes of Health, Bethesda, Md., 1987 and 1991).

An intact antibody means a tetrameric structure as described above having full-length immunoglobulin variable and constant regions. The variable region in an intact immunoglobulin is mature meaning that it lacks an immunoglobulin signal sequence. The terms “antibody” and “immunoglobulin” are used interchangeably. Binding fragments of intact antibodies, such as Fab, are also referred to as antibodies.

The hydrophobic amino acids are met, ala, val, leu, ile, and optionally cys, phe, pro, trp, and tyr as well. The hydrophilic amino acids are arg, asn, asp, gln, glu, his, lys, ser, and thr.

Specific binding between two entities means an affinity of at least 10⁶, 10⁷, 10⁸, 10⁹, or 10¹⁰ M⁻¹. Affinities greater than 10⁸ M⁻¹ are preferred.

Targets of interest for antibodies of the invention include growth factor receptors (e.g., FGFR, PDGFR, EGFR, NGFR, and VEGF) and their ligands. Other targets are G-protein receptors and include substance K receptor, the angiotensin receptor, the α- and β-adrenergic receptors, the serotonin receptors, and PAF receptor. See, e.g., Gilman, Ann. Rev. Biochem. 56:625-649 (1987). Other targets include ion channels (e.g., calcium, sodium, potassium channels), muscarinic receptors, acetylcholine receptors, GABA receptors, glutamate receptors, and dopamine receptors (see Harpold, U.S. Pat. No. 5,401,629 and U.S. Pat. No. 5,436,128). Other targets are adhesion proteins such as integrins, selectins, and immunoglobulin superfamily members (see Springer, Nature 346:425-433 (1990). Osborn, Cell 62:3 (1990); Hynes, Cell 69:11 (1992)). Other targets are cytokines, such as interleukins IL-1 through IL-13, tumor necrosis factors α & β, interferons α, β and γ, tumor growth factor Beta (TGF-β), colony stimulating factor (CSF) and granulocyte monocyte colony stimulating factor (GM-CSF). See Human Cytokines: Handbook for Basic & Clinical Research (Aggrawal et al. eds., Blackwell Scientific, Boston, Mass. 1991). Other targets are hormones, enzymes, and intracellular and intercellular messengers, such as, adenyl cyclase, guanyl cyclase, and phospholipase C. Other targets of interest are leukocyte antigens, such as CD20, and CD33. Drugs may also be targets of interest. Target molecules can be human, mammalian or bacterial. Other targets are antigens, such as proteins, glycoproteins and carbohydrates from microbial pathogens, both viral and bacterial, and tumors. Still other targets are described in U.S. Pat. No. 4,366,241.

Compositions or methods “comprising” one or more recited elements may include other elements not specifically recited.

A nucleic acid is operably linked when it is placed into a functional relationship with another nucleic acid sequence. For instance, a promoter or enhancer is operably linked to a coding sequence if it increases the transcription of the coding sequence. Operably linked means that the DNA sequences being linked are typically contiguous and, where necessary to join two protein coding regions, contiguous and in reading frame. However, because enhancers generally function when separated from the promoter by up to several kilobases or more, some polynucleotide elements may be operably linked but not contiguous.

As used herein, the term “predominantly” or variations such as “the predominant” or “which is predominant” in reference to a glycan species means the glycan species that has the highest mole percent (%) of total N-glycans after the glycoprotein has been treated with PNGase and released glycans analyzed by mass spectroscopy, for example, MALDI-TOF MS. In other words, the individual entity, such as a specific glycoform, which is present in greater mole percent than any other individual entity is “predominant.”. For example, if a composition consists of species A in 40 mole percent, species B in 35 mole percent and species C in 25 mole percent, the composition comprises predominantly species A.

Antibodies of the invention are typically isolated in substantially pure form from undesired contaminants including soluble proteins of the host cell. This means that an antibody is typically at least about 50% w/w (weight/weight) pure. Sometimes the antibodies are at least about 80% w/w and, more preferably at least 90 or about 95% w/w purity. Using conventional protein purification techniques, antibodies of at least 99% w/w purity can be obtained. An antibody is purified to essential homogeneity when under nonreducing conditions it runs as a single band on a gel and under reducing conditions it runs as two bands corresponding to component immunoglobulin heavy and light chains.

A lower eukaryotic host cell as used herein refers to any eukaryotic cell which ordinarily produces high mannose-containing N-glycans, and thus is meant to include some animal or plant cells and most typical lower eukaryotic cells, including yeast and filamentous fungal cells.

As used herein, the term “N-glycan” refers to an N-linked oligosaccharide, e.g., one that is attached by an asparagine-N-acetylglucosamine linkage to an asparagines residue of a polypeptide. N-glycans have a common pentasaccharide core of Man₃GlcNAc₂ (“Man” refers to mannose; “Glc” refers to glucose; and NAc” refers to N-acetyl; GlcNAc refers to N-acetylglucosamine). N-glycans differ with respect to the number of branches (antennae) comprising peripheral sugars (e.g., fucose and sialic acid) that are added to the Man₃GlcNAc₂ (“Man3”) core structure. N-glycans are classified according to their branched constituents (e.g., high mannose, complex or hybrid). A . . . “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose core”. The “trimannose core” is the pentasaccharide core having a Man3 structure. Complex N-glycans may also have galactose (“Gal”) residues that are optionally modified with sialic acid or derivatives (“NeuAc”, where “Neu” refers to neuraminic acid and “Ac” refers to acetyl). Complex N-glycans may also have intrachain substitutions comprising “bisecting” GlcNAc and core fucose (“fuc”). A “hybrid” glycan has at least one GlcNAc on the terminus of the 1,3 mannose arm of the trimannose core and zero or more mannoses on the 1,6 mannose arm of the trimannose core.

A spacer peptide in the present invention includes one or more proteolytic sites for cleavage of the light and heavy chains from the expressed fusion protein of the invention. A spacer peptide can also include a peptide linker. One function of the linker is to provide physical space and flexibility between the light chain and the heavy chain within the fusion protein of the invention. Optionally, the linker has other functions as well. For example, the linker can encode a functional secreted protein domain. It is not however necessary for either the immunoglobulin heavy or light chain to be fused with peptide sequences from a protein of the host in which the fusion protein is to be expressed. The presence of the signal sequence at the N-terminus of the fusion protein is sufficient to target the fusion protein to the appropriate cellular location for the processing and assembly steps described below to occur.

### DETAILED DESCRIPTION OF THE INVENTION

I. General

The invention provides methods suitable for expression of intact antibodies in a variety of cell types. Although the methods are especially suitable for expression in lower eukaryotic organisms, they can also be practiced in higher eukaryotic organisms and in bacteria. The methods involve expressing a nucleic acid encoding a single-chain antibody under the control of a single promoter. The single-chain antibody comprises an immunoglobulin light chain comprising a variable region and a constant region, a spacer peptide, and an immunoglobulin heavy chain comprising a variable region and a constant region. The spacer peptide includes at least one and usually two or more proteolytic cleavage sites. Although practice of the invention is not dependent on an understanding of mechanism, it is believed that expression of heavy and light chains in equimolar ratio due to linkage in a fusion protein, at least in part, overcomes difficulties previously experienced in obtaining assembly of intact antibodies in lower eukaryotes and bacteria. The spacer peptide can be removed by proteolytic cleavage before, during or after assembly of antibodies. Regardless of when removed, the spacer peptide does not prevent formation of heavy-light chain pairs or of tetrameric antibody. The end product of such expression and proteolytic cleavage is an antibody comprising at least one and preferably two pairs of heavy and light chains and lacking most or all spacer residues.

By selection of genetically modified lower eukaryotic host cells, appropriate glycosylation can also be achieved. A significant advantage of antibodies produced by these genetically modified hosts, is that the antibodies produced from such cells have one predominant glycoform and lack fucose (unless fucose is engineered into the host). The process can thus result in antibody compositions having many desired traits such as function and therapeutic activity similar to those of antibodies produced in mammalian cells without the inefficiencies and expense inherent from expression of mammalian cell cultures and with certain potentially advantageous characteristics such as uniformity of glycan structure.

II. Components of a Single-Chain Antibody

Antibodies of the invention are initially expressed as a fusion protein having several components. The components include at least the following from N-terminus to C-terminus, signal sequence, a first immunoglobulin chain, a spacer peptide and a second immunoglobulin chain. Either the light or heavy chain can be designated as the first chain, and the other as the second chain as a matter of arbitrary choice. The signal sequence directs the fusion protein down a pathway from the cytoplasm to within an organelle and/or across a cellular membrane (depending on the organism). In prokaryotes, a signal sequence directs the fusion protein across the cellular membrane to the periplasmic space in which antibody forms. In eukaryotes, a signal sequence directs the fusion protein from the cytoplasm to the endoplasmic reticulum to the Golgi, usually followed by secretion from the cell. As the fusion protein moves along this pathway, it is subject to several proteolytic processing steps, glycosylation, and folding steps described in more detail below.

Joined to the signal sequence are first and second immunoglobulin chains separated by a spacer peptide. A single signal sequence positioned on the N-terminal side of the first immunoglobulin chain serves to direct organelle targeting and/or secretion of the entire fusion protein including both the immunoglobulin heavy and light chains. Therefore, it is unnecessary and undesirable to include a second signal sequence between the spacer peptide and the second immunoglobulin chain.

Both the light and heavy chain have variable regions and constant regions. The variable regions are preferably complete variable regions, or if not complete they are at least, in combination, sufficient to confer specific binding to a target antigen. The heavy and light chain constant regions are at least sufficient to allow formation of a stable pair of heavy and light chains (e.g., a Fab fragments). Preferably, the light chain constant region is sufficient to allow formation of a pair of heavy and light chains, and the heavy chain constant region is sufficient to allow both formation of light chain-heavy chain pairs via intermolecular disulfide and noncovalent bonding of heavy and light chain constant regions, and formation of a tetrameric antibody comprising two pairs of heavy and light chains, the two pairs being associated through noncovalent and disulfide bonding of the respective heavy chain regions. Preferably both heavy and light chain constant regions are full length. Preferably, the antibody is a tetrameric antibody having an effector function such as complement fixation or antibody dependent cellular toxicity. The nature of effector function depends on isotype. For example, human isotopes IgG1 and IgG3 have complement activity and isotypes IgG2 and IgG4 do not.

Usually, the signal sequence at the N-terminus of the fusion protein serves both to direct the fusion protein transcript (mRNA) to the ER for further processing through the secretory pathway, i.e., through the Golgi and out of the cell. The signal sequence is often from the same organism as the cell in which antibody expression is to occur. However, such is not essential. Examples of signal sequences that can be used include the signal sequences from one of the following secreted proteins: S. cerevisiae alpha mating factor pre, Aspergillus alpha amylase, Aspergillus glucoamylase (GLA), human serum albumin (HSA), K. lactis inulinase (INU), S. cerevisiae invertase (INV), P. pastoris KAR2, S. cerevisiae killer toxin pre (KILM), P. pastoris phosphatase I (PHOI), S. cerevisiae alpha mating factor prepro, P. pastoris alpha mating factor preproKR, and chicken lysozyme (ChicLys). The signal sequence is typically cleaved from the fusion protein transcript upon intracellular processing.

The immunoglobulin heavy and light chains can be from any type of antibody. Usually the antibody is a monoclonal antibody although polyclonal antibodies can also be expressed recombinantly (see, e.g., U.S. Pat. No. 6,555,310). Examples of antibodies that can be expressed include mouse or murine antibodies, chimeric antibodies, humanized antibodies, veneered antibodies and human antibodies. Chimeric antibodies are antibodies whose light and heavy chain genes have been constructed, typically by genetic engineering, from immunoglobulin gene segments belonging to different species (see, e.g., Boyce et al., Annals of Oncology 14:520-535 (2003)). For example, the variable (V) segments of the genes from a mouse monoclonal antibody may be joined to human constant (C) segments. A typical chimeric antibody is thus a hybrid protein consisting of the V or antigen-binding domain from a mouse antibody and the C or effector domain from a human antibody.

Humanized antibodies have variable region framework residues substantially from a human antibody (termed an acceptor antibody) and complementarity determining regions substantially from a mouse-antibody, (referred to as the donor immunoglobulin). See Queen et al., Proc. Natl. Acad. Sci. USA 86:10029-10033 (1989) and WO 90/07861, U.S. Pat. No. 5,693,762, U.S. Pat. No. 5,693,761, U.S. Pat. No. 5,585,089, U.S. Pat. No. 5,530,101 and Winter, U.S. Pat. No. 5,225,539. The constant region(s), if present, are also substantially or entirely from a human immunoglobulin. Antibodies can be obtained by conventional hybridoma approaches, phage display (see, e.g., Dower et al., WO 91/17271 and McCafferty et al., WO 92/01047), use of transgenic mice with human immune systems (Lonberg et al., WO93/12227 (1993)), among other sources. Nucleic acids encoding immunoglobulin chains can be obtained from hybridomas or cell lines producing antibodies, or based on immunoglobulin nucleic acid or amino acid sequences in the published literature.

The light and heavy chains are separated by a spacer peptide. The peptide joins the C-terminus of the chain linked to the signal sequence to the N-terminus of the other chain. The spacer peptide contains at least one, and preferably two or more proteolytic cleavage sites cleavable by a protease which is a separate molecule from the fusion protein. That is, in the present invention cleavage of the proteolytic cleavage sites is an intermolecular reaction rather than intramolecular. The peptide spacer lacks self-processing cleavage site(s) cleavable by an intramolecular autocatalytic mechanism effected by the fusion protein, or particularly the spacer peptide component thereof. An example of a self-processing cleavage site is described by de Felipe et al., J. Biol. Chem. 278, 11441-11448 (2003). Such self-cleavable sites cleave the peptide spacer prematurely before other processing steps such as organelle targeting and antibody assembly have occurred. As a result of such premature cleavage, at least in lower eukaryotes, the same signal peptide cannot target both immunoglobulin chains to the endoplasmic reticulum for further proteolytic processing.

If the peptide spacer contains two or more proteolytic cleavage sites, the proteolytic sites can be the same or different and cleaved by the same or different protease. Usually, if the peptide spacer contains two or more proteolytic cleavage sites, all are cleaved by the same protease. However, if the peptides spacer contains two or more proteolytic cleavage sites cleaved by different proteases, the different proteases are all separate molecules from the fusion protein. That is, none of the proteolytic cleavage sites is a self-cleaving proteolytic cleavage site.

Optionally, the spacer peptide consists of one or more proteolytic cleavage sites in tandem. Alternatively, a spacer peptide can consist of a linker flanked by a pair of proteolytic cleavage sites. The proteolytic site(s) are arranged such that cleavage at those sites separates the heavy and light chains from being components of the same fusion protein and releases them as separate chains. The chains are separated in the sense that they are not linked by peptide bonds. However, the chains can be linked by intermolecular disulfide bonding and noncovalent bonding between heavy and light chains. Preferably, the proteolytic cleavage sites are arranged to separate the immunoglobulin heavy and light chains from most or all of the spacer peptide without cleaving any immunoglobulin residues.

The choice of proteolytic site depends in part on the choice of the host cell. In some methods, the site is one that is cleaved by a protease naturally present in the desired host cell. In other methods, the proteolytic site is cleaved by a protease introduced into the desired host cell by genetic engineering. The proteolytic cleavage site is preferably chosen such that the same site is not present on the immunoglobulin heavy or light chain. A preferred proteolytic cleavage site for yeast host cells is the protease Kex2p. This enzyme cleaves on the C-terminal side of the amino acid pair KR. Preferably, the pair is preceded on the N-terminal side by one or two hydrophobic residues, such as LV, or hydrophilic residues such as RH or KH. Optionally, an arginine residue is also present as in RLV. A preferred format for a fusion protein is signal sequence—Light Chain—LVKRlinkerRLVKR—Heavy Chain. Kex2p cleavage occurs after both of the KR residue pairs. Degradation by another protease removes the LVKR residues leaving separated immunoglobulin light and heavy chains and no intervening spacer residues.

In addition to Kex2p, other known endogenous yeast proteolytic enzymes include: Kex1p and Ste13p both located in the late Golgi, and BplIp, CPYp and Pep4p located in the vacuole (lysosome). The cleavage sequences for these enzymes have been disclosed previously (JCB, 1992, 119: 1459-1468; Yeast, 1994, 10: 801-810; FEMS Microbiol. Lett, 1995, 130: 221-229; Ann. Rev Genet. 1984, 18: 233-270.). When expression of endogenous Kex2p is low, addition of EAEA after either or both Kex2p sites in the linker (e.g., LVKREAEA) improves cleavage by Kex2p and/or Ste13p. Addition of EAEA is particularly useful after the second Kex2p site. Optionally, Kex2p from S. cerevisiae or P. pastoris is overexpressed in the host cell under the control of an inducible promoter to improve cleavage.

For in vivo cleavage in mammalian host cells, the proteolytic sites and enzymes which can be used may include: Factor Xa, thrombin, signal peptidase I and furin. These proteolytic enzymes are well known in the art.

Other than the proteolytic cleavage site(s), the composition of the spacer peptide is not critical. If the spacer peptide consists of more than the proteolytic cleavage site and contains a linker, a variety of linkers suitable for expression of single chain antibodies and principles for their design are known in the art (see Huston et al., Proc. Natl. Acad. Sci. USA 85 5879-5883 (1988); Bird et al., Science 242, 423-426 (1988); U.S. Pat. No. 4,946,778, U.S. Pat. No. 5,132,405 and U.S. Pat. No. 5,482,858, U.S. Pat. No. 5,258,498). In general, the linker should be of sufficient length and flexibility to permit intramolecular association of heavy and light chains of the same fusion protein or intermolecular associations between heavy and lights chains on different fusion proteins. Glycines and/or serines are particularly suitable for inclusion in a linker. Suitable lengths range from about 0 to 100 amino acids. Total spacer peptide lengths of 15 amino acids or greater (including proteolytic cleavage sites) favor intramolecular bonding of heavy and light chains. Shorter spacer peptide lengths favor intermolecular bonding. One example of a suitable linker is a four glycines (G) and one serine (S) motif repeated 16 times followed by proline (P) and five more glycines residues just before the C-terminal proteolytic cleavage site [(GGGGS)₁₆PGGGGG]. The proline, which has a boxy ring structure, provides a stable hinge-like structure to the flexible glycine linker. Another example of a flexible linker is a repeat of at least three units of gly, ser [e.g. GSGSGS]. As a further alternative, the spacer can have a chain of proteolytic cleavage sites (e.g., 5-30 Kex2 sites) in tandem.

As well as the above components, the peptide spacer can include a secreted protein domain. The secreted protein domain can function in at least two roles. First, it can enhance and/or promote the secretion and assembly of the downstream chain(s). Second, in lower eukaryotic hosts, this secreted protein domain can be used to saturate O-glycosylation enzymes, thereby reducing the extent of the non-human O-glycosylation present on the expressed heavy and light chains.

It is not however necessary for either the immunoglobulin heavy or light chain to be fused with peptide sequences from a protein of the host in which the fusion protein is to be expressed. The presence of the signal sequence at the N-terminus of the fusion protein is sufficient to target the fusion protein to the appropriate cellular location for the processing and assembly steps described below to occur. Thus, for example, the signal sequence can be directly fused to the first immunoglobulin chain without intervening amino acids, and the spacer peptide can be fused directly to the second immunoglobulin chain, and the spacer peptide can itself be free of any peptide sequences from a host protein.

A further optional component of the fusion protein is a peptide tag to assist in identification or purification of the fusion protein or antibody resulting from processing of the same. Such a tag can be placed N-terminal of the first immunoglobulin chain, within or joined to one end of the spacer peptide, or C-terminal of the second immunoglobulin chain. An example of a tag is the FLAG™ system (Kodak). The FLAG™ molecular tag consists of an eight amino acid FLAG peptide marker that is linked to the target binding moiety. Antibodies suitable for use with the FLAG peptide marker. Another example is a polyhistidine tag which can be bound by metal chelate ligands (see Hochuli in Genetic Engineering: Principles and Methods (ed. J K Setlow, Plenum Press, NY), Ch. 18, pp. 87-96 and maltose binding protein (Maina, et al., Gene 74:365-373 (1988)). Several other peptide tags are known and readily available.

III. Nucleic Acid Encoding the Fusion Protein

Fusion proteins described above are encoded by nucleic acids. The nucleic acids can be DNA or RNA, preferably DNA. The nucleic acid encoding the fusion protein is operably linked to regulatory sequences that allow expression of the fusion protein. Such regulatory sequences include a promoter and optionally an enhancer upstream, or 5′, to the nucleic acid encoding the fusion protein and a transcription termination site 3′ or down stream from the nucleic acid encoding the fusion protein. The nucleic acid also typically encodes a 5′ UTR region having a ribosome binding site and a 3′ untranslated region. The nucleic acid is often a component of a vector replicable in cells in which the antibody is expressed. The vector can also contain a marker to allow recognition of transformed cells. However, some cell types, particularly yeast, can be successfully transformed with a nucleic acid lacking extraneous vector sequences.

Nucleic acids encoding immunoglobulin light and heavy chains can be obtained from several sources. cDNA sequences can be amplified from hybridomas or other cell lines expressing antibodies using primers to conserved regions (see, e.g., Marks et al., J. Mol. Biol. 581-596 (1991)). Nucleic acids can also be synthesized de novo based on sequences in the scientific literature. Nucleic acids can also be synthesized by extension of overlapping oligonucleotides spanning a desired sequence (see, e.g., Caldas et al., Protein Engineering, 13, 353-360 (2000)).

IV. Host Cells

Lower eukaryotes are preferred for expression of antibodies because they can be economically cultured, give high yields, and when appropriately modified are capable of suitable glycosylation. Yeast and filamentous fungus particularly offer established genetics allowing for rapid transformations, tested protein localization strategies and facile gene knock-out techniques. Suitable vectors have expression control sequences, such as promoters, including 3-phosphoglycerate kinase or other glycolytic enzymes, and an origin of replication, termination sequences and the like as desired. Vectors can also include segments flanking the nucleic acid encoding the fusion protein of the invention, which segments are capable of recombining with selected regions of the host chromosome, thereby targeting a nucleic acid to a chromosomal location favoring expression.

Various yeasts such as: Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, and Candida albicans are preferred for cell culture because they are able to grow to high cell densities and secrete large quantities of recombinant protein. Likewise, filamentous fungi, such as Aspergillus nidulans, Aspergillus niger, Aspergillus oryzae, Trichoderma reesei, Chrysosporium lucknowense, Fusarium sp., Fusarium gramineum, Fusarium venenatum and Neurospora crassa (see, e.g., U.S. Pat. No. 5,364,770, EP 214,914 and WO 90/15860) and others can be used to produce glycosylated antibodies of the invention at an industrial scale.

Lower eukaryotes, particularly yeast and filamentous fungus, can be genetically modified so that they express antibodies (or other proteins) in which the glycosylation pattern is human-like or humanized. Such can be achieved by eliminating selected endogenous glycosylation enzymes and/or supplying exogenous enzymes as described by Gerngross et al., US 20040018590; Hamilton et al., 2003, Science, 301: 1244-1246). For example, a host cell can be selected or engineered to be depleted in α-1,6-mannosyl transferase activities which would otherwise add mannose residues onto the N-glycan on a glycoprotein.

Such a host cell can additionally or alternatively be engineered to express one or more enzymes which enable the production of a complex carbohydrate structure (and its synthetic intermediates) in vivo. Such an enzyme can be targeted to a host subcellular organelle in which the enzyme has optimal activity, e.g., by means of signal peptide not normally associated with the enzyme. Such host cells can also be modified to express a sugar nucleotide transporter and/or a nucleotide diphosphatase enzyme. The transporter and diphosphatase improve the efficiency of engineered glycosylation steps, by providing the appropriate substrates for the glycosylation enzymes in the appropriate compartments, reducing competitive product inhibition, and promoting the removal of nucleotide diphosphates.

Another advantage of these engineered host cells is that they can be used to produce antibody compositions with predominantly one glycoform structure, which lacks fucose, unless the fucose in specifically engineered in. Similar to the role of glycosylation in other glycoproteins, the oligosaccharide side chains of antibodies affect this glycoprotein's function. For example, it has been shown that an antibody composition having decreased fucosylated N-linked glycan enhances binding to human FcγRIII and therefore enhances antibody-dependent cellular cytotoxicity (ADCC) (Shields et al., 2002, J. Biol Chem, 277: 26733-26740; Shinkawa et al., 2003, J. Biol. Chem. 278: 3466-3473). Homogenous forms of fucosylated G2 (Gal₂GlcNAc₂Man₃GlcNAc₂) IgGs made in CHO cells increase CDC activity to a greater extent than heterogeneous antibodies (Raju, 2004, US Pat. Appl. No. 2004/0136986).

Practice of the methods of the invention in appropriately engineered lower eukaryotic host cells results in predominantly homogenous glycoforms. That is, the antibodies produced by such a cell have a predominant N-glycan structure at corresponding N-glycosylation sites. Additionally, the glycans produced in yeast and filamentous fungal hosts disclosed in the present invention, naturally lack fucose. Accordingly, the engineered host cells of the present invention are capable of producing antibodies having complex N-glycans predominantly of one glycoform structure and lacking fucose (unless the fucose is specifically engineered in). In one embodiment, the present invention provides an antibody composition produced by the process of the present invention comprising predominantly one glycan structure, wherein the predominant glycan structure is present at a level that is at least about 5 mole percent more than the next predominant glycan structure of the antibody composition.

In another embodiment, the present invention provides an antibody composition produced by the process of the present invention comprising predominantly one glycan structure, wherein the predominant glycan structure is present at a level that is at least about 10-25 mole percent more than the next predominant glycan structure of the antibody composition.

In another embodiment, the present invention provides an antibody composition produced by the process of the present invention comprising predominantly one glycan structure, wherein said predominant glycan structure is present at a level that is at least about 25-50 mole percent more than the next predominant glycan structure of the antibody composition.

In another embodiment, the present invention provides an antibody composition produced by the process of the present invention comprising predominantly one glycan structure, wherein said predominant glycan structure is present at a level that is at least about 50 mole percent more than the next predominant glycan structure of the antibody composition.

Prokaryotic hosts that can be used to express antibodies include E. coli, bacilli, such as Bacillus subtilis, and other enterobacteriaceae, such as Salmonella, Serratia, and various Pseudomonas species. Prokaryotes have some of the advantages of lower eukaryotes in terms of ease of culture, but are not able to carry out appropriate glycosylation. In prokaryotic hosts, one can also make expression vectors, which typically contain expression control sequences compatible with the host cell (e.g., an origin of replication). In addition, a variety of well-known promoters are present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation.

Plants and plant cell cultures may be used for expression antibodies of the invention. (Larrick & Fry, Hum. Antibodies Hybridomas 2(4):172-89 (1991); Benvenuto et al., Plant Mol. Biol. 17(4):865-74 (1991); Durin et al., Plant Mol. Biol. 15(2):281-93 (1990); Hiatt et al., Nature 342:76-8 (1989). Preferable plant hosts include, for example: Arabidopsis, Nicotiana tabacum, Nicotiana rustica, and Solanum tuberosum.

Insect cell culture can also be used to produce antibodies of the invention, typically using a baculovirus-based expression system (see Putlitz et al., Bio/Technology 8:651-654 (1990)).

Although not as economical to culture as lower eukaryotes and prokaryotes, mammalian tissue cell culture can also be used to express and produce the polypeptides of the present invention (see Winnacker, From Genes to Clones (VCH Publishers, NY, 1987). Suitable hosts include CHO cell lines, various COS cell lines, HeLa cells, preferably myeloma cell lines and the like, or transformed B-cells or hybridomas. Expression vectors for these cells can include expression control sequences, such as an origin of replication, one or more promoters, one or more enhancers (Queen et al., Immunol. Rev. 89:49-68 (1986), and necessary processing information sites, such as ribosome binding sites, RNA splice sites, polyadenylation sites, and transcriptional terminator sequences. Expression control sequences may be promoters derived from immunoglobulin genes, SV40, Adenovirus, bovine Papilloma Virus, cytomegalovirus and the like. Preferred promoters may be constitutive or inducible. Generally, a selectable marker, such as a neoR expression cassette, is included in the expression vector.

The nucleic acid encoding the immunoglobulin chains to be expressed can be transferred into the host cell by conventional methods, which vary depending on the type of cellular host. For example, calcium chloride transfection is commonly used for prokaryotic cells, whereas calcium phosphate treatment, protoplast fusion, natural breeding, lipofection, biolistics, viral-based transduction, or electroporation can be used for other cellular hosts. Tungsten particle ballistic transgenesis is preferred for plant cells and tissues. (See, generally, Maniatis et al., Molecular Cloning: A Laboratory Manual (Cold Spring Harbor Press, 1982), which is incorporated herein by reference in its entirety for all purposes.). Preferably, nucleic acids are stably maintained in host cells, either as episomes, or integrated into the genome of host cells.

Once expressed, antibodies of the present invention can be purified according to standard procedures of the art, including ammonium sulfate precipitation, affinity columns, column chromatography, gel electrophoresis and the like (see, generally, Scopes, R., Protein Purification (Springer-Verlag, N.Y., 1982), which is incorporated herein by reference in its entirety for all purposes). Substantially pure immunoglobulins of at least about 90 to 95% homogeneity are preferred, and 98 to 99% or more homogeneity most preferred, for pharmaceutical uses. Once purified, partially or to homogeneity as desired, the polypeptides can then be used therapeutically (including extracorporeally) or in developing and performing assay procedures, immunofluorescent stainings, and the like. (See, generally, Immunological Methods, Vols. I and II (Lefkovits and Pernis, eds., Academic Press, NY, 1979 and 1981).

V. Antibody Expression, Processing, Assembly, and Secretion

A nucleic acid encoding a fusion protein comprising a signal sequence, immunoglobulin light chain, spacer peptide and immunoglobulin heavy chain, is initially expressed as the fusion protein. The fusion protein is then subject to a series of processing and folding events that produce an antibody comprising a heavy and light pair in which the chains are intermolecularly but not intramolecularly associated. These events can include the targeting of the fusion protein to an organelle and/or secretion from the host mediated by the signal sequence, the processing of the signal sequence, the intra or intermolecular association of immunoglobulin heavy and light chains to form a heterodimeric pair, the pairing of two heterodimers to form a tetramer, formation of disulfide bonds, glycosylation, and the cleavage of proteolytic site(s) in the spacer peptide so that the heavy and light chain are no longer components of the same peptide chain. The order and precise nature of these events may vary depending on culture conditions, the nature of the construct, the signal sequence, spacer peptide and the host cell. An understanding of mechanism is not required for practice of the invention.

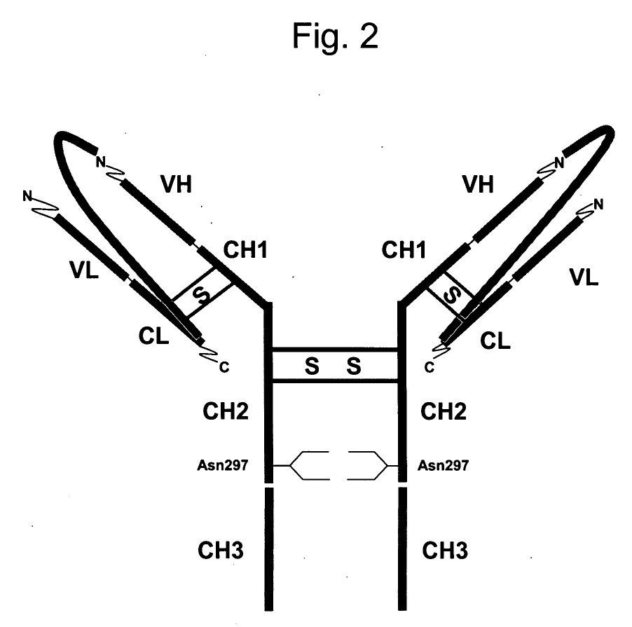

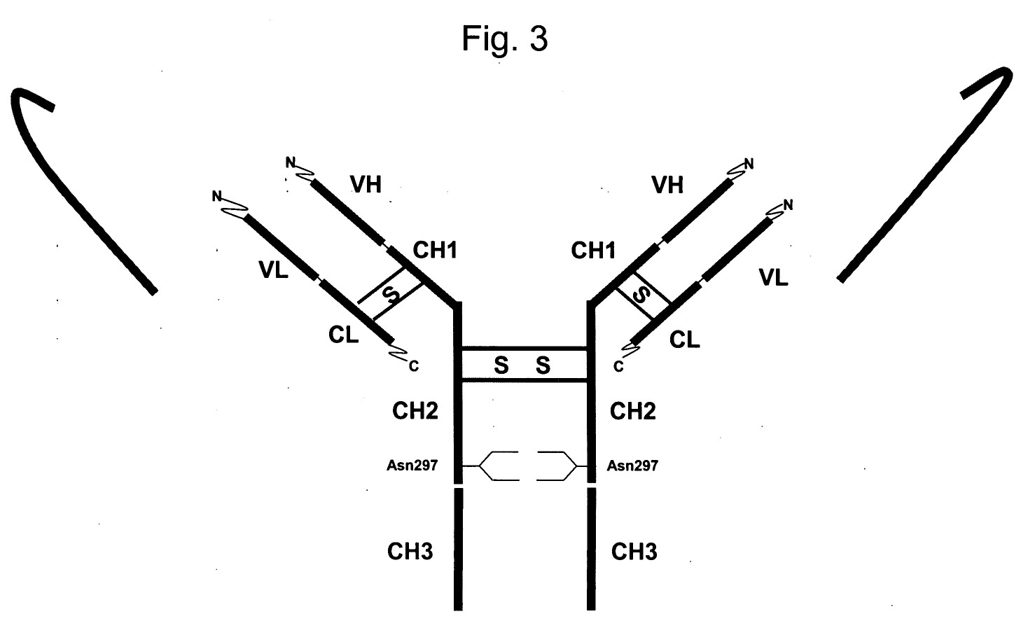

FIG. 2 shows an intermediate in one possible sequence of post-translational modifications of the fusion protein. The figure shows a tetrameric antibody formed by association of two fusion protein. In each fusion protein, the immunoglobulin light and heavy chains are intramolecularly associated by noncovalent bonding between the light and heavy chain variable regions and a disulfide bond between the constant regions. The two fusion proteins are held together by noncovalent and disulfide interactions between the Fc regions of the respective heavy chains. Residue Asn 297 of the heavy chain constant region is glycosylated in both fusion proteins. FIG. 3 shows the same antibody after cleavage of proteolytic site(s) in the spacer peptide. The conformation of the tetrameric antibody is unchanged except that the heavy and light chains which were part of the same fusion protein and intermolecularly associated are now separate chains and intermolecularly associated. The length of spacer peptide, if any, that remains attached to the antibody depends on the location of the cleavage sites in the spacer peptide, and the action of any exopeptidases in degrading any residual spacer peptide. It has been found that a linker flanked by the Kex2p sites LVKR and RLVKR is removed by Kex2p cleavage after the R residue and subsequent exoprotease activity removes LVKR from the first (N-terminal) proteolytic cleavage to give a substantially homogeneous antibody product lacking spacer peptide residues. Insofar as there is heterogeneity in the antibody product, the desired form of antibody can be separated from other cleavage products by further cleavage performed in vitro and/or hydrophobic interaction chromatography (HIC) and cation exchange chromatography (see Example 3).

Although cleavage of proteolytic site(s) preferably occurs in vivo, it can also be performed in vitro. Antibody is secreted or otherwise released from host cells and treated with a protease known to cleave the proteolytic sites. Several proteolytic sites can be used. In addition to those which are found in the host cell, other proteolytic sites can be introduced into the fusion protein construct. These sites can be cleaved either by introducing the proteolytic enzyme into the host cell for in vivo cleavage, or by expressing and purifying the uncleaved fusion protein and cleaving the linker region in an in vitro reaction. All proteolytic enzymes which have been disclosed to work in vivo, can be purified or purchased for use in an in vitro cleavage reaction.

For in vitro Kex2p cleavage, Kex2p is first purified (PNAS, 1992, 89: 922-926) and then both Kex2p and the Protein A-purified uncleaved antibody (Example X) are incubated as described (JBC, 1995, 270: 3154-3159). This in vitro cleavage is then followed by a second Protein A chromatography to isolate the heavy and light chains from the Kex2p protein.

Usually the signal sequence causes the protein fused to the signal sequence to be secreted from the host cell. If secretion does not occur, antibody can be released from the host cell by induced lysis. Lysis can be induced by sonication, freeze-thaw cycling or treatment with lysozyme among other methods.

VII. Variation

As discussed above, the protease responsible for cleaving proteolytic site(s) in the spacer peptide can be naturally present in the cell, supplied exogenously to the cell, or provided in vitro. In a variation, the protease is targeted by selection of an appropriate signal peptide to an organelle in the secretory pathway. Such targeting can be achieved by selection of the signal peptide linked to the protease. Such targeting can be used both for a protease naturally found with a host cell (e.g., Kex2p in yeast cells) or a protease supplied exogenously. Targeting affects the timing at which proteolytic cleavage occurs relative to other processing steps. For example, if a protease is targeted to an early organelle of secretion pathway proteolytic processing occurs earlier relative to folding of the fusion protein. In such circumstances, proteolytic processing is more likely to be complete. For example, in natural yeast cells, most Kex2p processing occurs in the late Golgi. By the time fusion protein reaches the late Golgi, antibody assembly is substantially complete. By expressing additional Kex2p linked to an ER-targeting peptide, Kex2p is expressed in the endoplasmic reticulum. In this case, the proteolytic cleavage sites are processed before substantial antibody folding has occurred leading to more efficient cleavage.

The yield of antibody produced by methods of the invention is preferably at least 50 mg/liter culture medium, and more preferably at least 100 mg/l, 500 mg/L, 1 g/L or 2 g/L culture medium.

VIII. Pharmaceutical Compositions

Antibodies of the invention can be incorporated into pharmaceutical compositions comprising the antibody as an active therapeutic agent and a variety of other pharmaceutically acceptable components. See Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation can also include other carriers, adjuvants, or nontoxic, nontherapeutic, nonimmunogenic stabilizers and the like.

Pharmaceutical compositions for parenteral administration are sterile, substantially isotonic, pyrogen-free and prepared in accordance with GMP of the FDA or similar body. Antibodies can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249, 1527 (1990) and Hanes, Advanced Drug Delivery Reviews 28, 97-119 (1997).

IX. Diagnostic Products

Antibodies of the invention can also be incorporated into a variety of diagnostic kits and other diagnostic products such as an array. Antibodies are often provided prebound to a solid phase, such as to the wells of a microtiter dish. Kits also often contain reagents for detecting antibody binding, and labeling providing directions for use of the kit. Immunometric or sandwich assays are a preferred format for diagnostic kits (see U.S. Pat. Nos. 4,376,110, 4,486,530, 5,914,241, and 5,965,375). Antibody arrays are described by e.g., U.S. Pat. No. 5,922,615, U.S. Pat. No. 5,458,852, U.S. Pat. No. 6,019,944, and U.S. Pat. No. 6,143,576.

### EXAMPLES

1. Design of Fusion Protein and Nucleic Acid Encoding Same

A fusion protein for expressing antibody anti-DX was designed as follows:

MVAWWSLFLYGLQVAAPALA [SEQ ID NO:1] mature light chain

LVKRGGGGSGGGGSGGGGSGGGGSGGGGSGGGGSGGGGSGGGASGGGGSGGGGS GGGGSGGGGSGGGGSGGGGSGGGGSGGGGSPGGGGGRLVKR [SEQ ID NO:2] mature heavy chain.

The alpha-amylase signal sequence is shown in italics. A spacer peptide between the mature light and heavy chains is shown underlined. The DNA sequence encoding the signal sequence is:

[SEQ ID NO: 3]

ATG GTC GCTTGG TGG TCT TTG TTT CTG TAC GGT CTT CAG
GTC GCT GCA CCT
GCT TTG GCT

DNA encoding the variable region of the light chain of anti-DX antibody was synthesized by PCR overlap and a Mly1 site was added upstream of the first immunoglobulin chain. DNA encoding a light chain constant region of an IgG1 was ordered from GeneArt Inc. DNA encoding the whole light chain was prepared by PCR overlap extension and cloned a pCR2.1 topo vector. The whole light chain had a Mly1 site at 5′ end and a Not1 site 3′ of the stop codon. The DNA encoding the whole light chain was ligated with DNA encoding the alpha-amylase signal sequence into pPICZA vector. The alpha-amylase signal sequence was synthesized from overlapping oligonucleotides. The signal sequence has a Kozak sequence in front of ATG and also has an EcoR1 site at the 5′ terminal end. pPICZA was digested with EcoR1 and Not1. The alpha-amylase signal sequence has an EcoR1 site overhanging at the 5′ and 3′ termini which were blunt Ended. The light fragment was digested by Mly1 and Not1 from pCR2.1 topo vector and these three pieces were ligated together. The resulting plasmid is pDX398.

DNA encoding the Heavy and light chain variable regions of anti-DX antibody was synthesized using overlapping oligonucleotides. DNA encoding the heavy chain constant region of IgG1 was ordered from GeneArt Inc. DNA encoding the intact heavy chain was then prepared by overlap PCR and cloned into pCR2.1 topo vector, generating the plasmid pDX344.

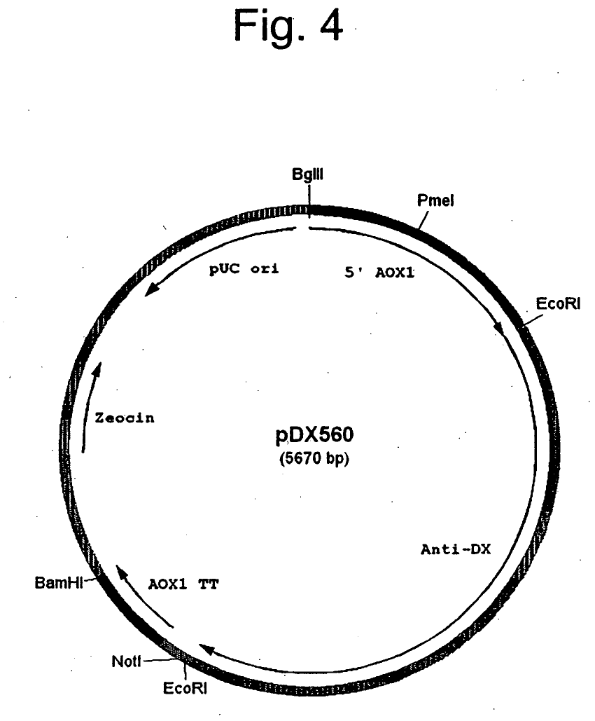

The vector pPICZA was cut by EcoR1 and Not1. A light chain fragment including the alpha-amylase signal sequence was digested by EcoR1 and Sph1 which is located at the end of the light chain constant region. A linker including part of the light chain constant region was synthesized by overlapping oligonucleotides, with the 5′ terminal oligonucleotide containing one Sph1 site. Plasmid pCR2.1topo with heavy chain was digested with Mly1 and Not1 and the band were recovered from an agarose gel. Four fragments were ligated to give the vector shown in FIG. 4.

Plasmid pDX560 was linearized by Pme1 and transformed into several Pichia pastoris strains—e.g. strain YAS306 transformed with α-mannosidase I, II, N-acetylglucosamine transferase I, II and galactosyl transferase (Gerngross et al., US 20040018590; Hamilton et al., Nature, 2003, 301: 1244-1246) in a background lacking α1,6 mannosyltransferase (Δoch1) (Choi et al., 2003, 100: 5022-5027) mannosylphosphate (Δpnol, Δmnn4b) (U.S. patent application Ser. No. 11/020808) and the alpha mannosidase resistant 2 (Δamr2) gene. (U.S. Appl. No. 60/566,736 and 60/620,186).

2. Culture Conditions for P. pastoris Strains—e.g. YAS306

A 10-ml culture of buffered glycerol-complex medium (BMGY) consisting of 1% yeast extract, 2% peptone, 100 mM potassium phosphate buffer (pH 6.5), 1.34% yeast nitrogen base, 4×10⁻⁵% biotin, and 1% glycerol was inoculated with a fresh colony of YAS306 containing plasmid pDX560 and grown for 2 days. The culture was then transferred into 100 mls of fresh BMGY in a 1 liter flask for 1 day. This culture is then centrifuged and the cell pellet washed with BMMY (buffered minimal methanol: same as BMGY except 0.5% methanol instead of 1% glycerol). The cell pellet was resuspended in BMMY to a volume ⅕ of the original BMGY culture and placed in 1.5 liter fermentation reactor for 24 h. The secreted protein was harvested by pelleting the biomass by centrifugation and transferring the culture medium to a fresh tube. The supernatant containing the secreted antibody was collected for purification.

3. Purification of Fusion Protein

Monoclonal antibodies were captured from the culture supernatant using a Streamline Protein A column. Antibodies were eluted in Tris-Glycine pH 3.5 and neutralized using 1M Tris pH 8.0. Further purification was carried out using hydrophobic interaction chromatography (HIC). The specific type of HIC column depends on the antibody. For the anti-DX antibody, a phenyl Sepharose column was used with 20 mM Tris (7.0), 1M (NH₄)₂SO₄ buffer and eluted with a linear gradient buffer of 1M to 0M (NH₄)₂SO₄. The antibody fractions from the phenyl Sepharose column were pooled and exchanged into 50 mM NaOAc/Tris pH 5.2 buffer for final purification through a cation exchange (SP Sepharose Fast Flow) (GE Healthcare) column. Antibodies were eluted with a linear gradient using 50 mM Tris, 1M NaCl (pH 7.0)

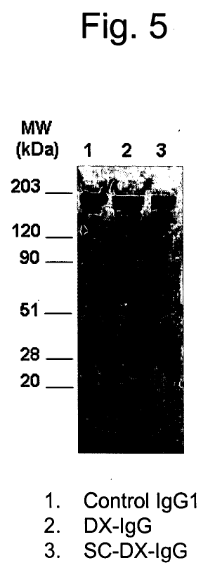

FIG. 5 is a Coomassie blue non-reducing SDS-PAGE gel showing the heavy- and light chains migrating at approximately 150 kDa as expected for a tetrameric assembly of heavy and light chains (Antibodies: A Laboratory Manual, Cold Spring Harbor Laboratory, Chapter 14, 1998; Monoclonal Antibodies: Principles and Practice, Academic Press Limited, 1996). Lane 1 shows a commercially prepared IgG control, Lane 2 shows DX-IgG produced in P. pastoris using the tradition method for recombinant antibody production in which expression of the heavy and light chains are driven by separate promoters and Lane 3 shows SC-DX-IgG produced in P. pastoris by the single promoter method according to this invention.

SDS-PAGE Tris-HCl gels (4-20% gradient and 15%) were purchased from Bio-Rad Laboratories and the molecular weight markers Bio-Rad Prestained SDS-PAGE Broad Range Molecular Weight Standards. Coomassie blue protein stain was purchased from Bio-Rad.

In accordance with a known method (Nature, 227, 680, 1970), 20 μg of anti-DX antibody was produced and purified as disclosed in above examples and subject to SDS-PAGE to analyze the molecular weight and degree of purification. As shown in FIG. 5, a single band of about 150 kDa in molecular weight was present under non-reducing conditions. This molecular weight coincides with the reports stating that an IgG antibody has a molecular weight of about 150 kDa under non-reducing conditions and is degraded into heavy chains having a molecular weight of about 50 kDa and light chains having a molecular weight of about 25 kDa under reducing conditions due to cutting of the disulfide bond in the molecule (Antibodies: A Laboratory Manual, Cold Spring Harbor Laboratory, Chapter 14, 1998; Monoclonal Antibodies: Principles and Practice, Academic Press Limited, 1996).

4. Antigen Binding ELISA Assay

High binding microtiter plates (Costar) are coated with 10 ug of antigen in PBS, pH 7.4 and incubate over night at 4° C. Buffer is removed and blocking buffer (3% BSA in PBS), is added and then incubated for 1 hour at room temperature. Blocking buffer is removed and the plates are washed 3 times with PBS. After the last wash, increasing amounts of purified antibody are added from 0.2 ng to 100 ng and incubated for 1 hour at room temperature. Plates are then washed with PBS+0.05% Tween20. After last wash, anti-human Fc-HRP is added in a 1:2000 PBS solution, and then incubated for 1 hour at room temperature. Plates are then washed 4 times with PBS-Tween20. Plates are analyzed using TMB substrate kit following manufacturer's instructions (Pierce Biotechnology).

5. Fc Receptor Binding Assay

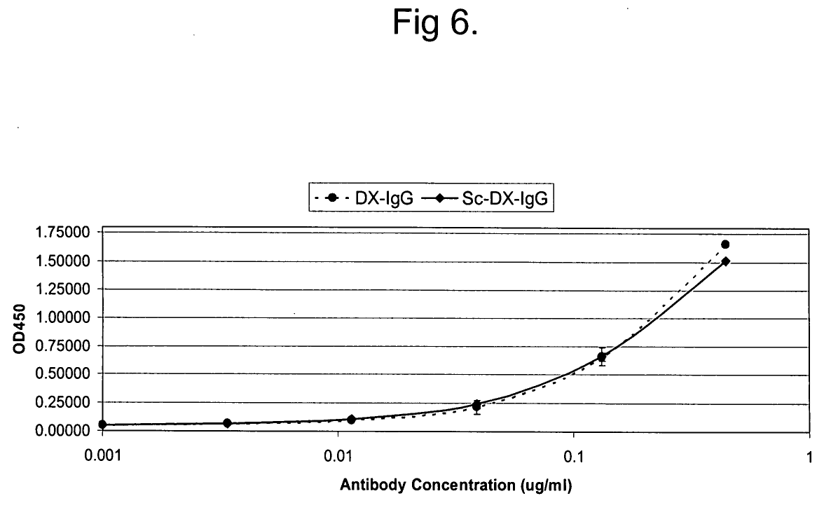

Fc receptor binding assays for FcγRI, FcγRII, FcγRIII and FcγRn were carried out according to the protocols previously described (JBC, 2001, 276: 6591-6604). For FcγRIII binding: FcγRIII fusion proteins at 1 μg/ml in PBS, pH 7.4, are coated onto ELISA plates (Nalge-Nunc, Naperville, Ill.) for 48 h at 4° C. Plates are blocked with 3% bovine serum albumin (BSA) in PBS at 25° C. for 1 h. anti-DX IgG1 dimeric complexes are prepared in 1% BSA in PBS by mixing 2:1 molar amounts of anti-DX IgG and HRP-conjugated F(Ab′)2anti-F(Ab′)2 at 25° C. for 1 h. Dimeric complexes were then diluted serially at 1:2 in 1% BSA/PBS and coated onto the plate for 1 hour at 25° C. The substrate used is 3,3′,5,5′-tetramethylbenzidine (TMB) (Vector Laboratories). Absorbance at 450 nm is read following instructions of the manufacturer (Vector Laboratories). FIG. 6 compares binding of DX IgG with SC DX IgG produced in P. pastoris.

6. Binding of Antibody to ErbB2/Fc Antigen

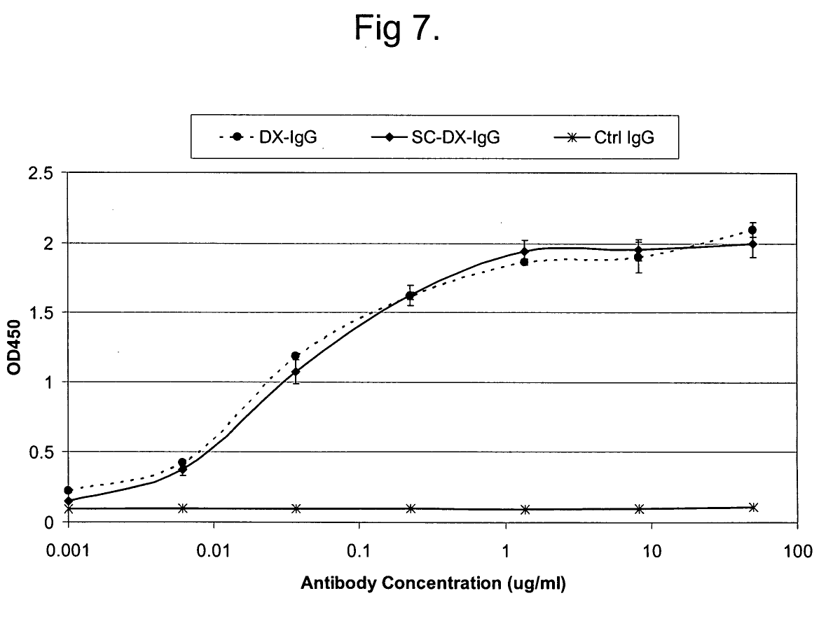

ELISA plate (Corning Costar) was coated with 100 ul/well of recombinant human ErbB2/Fc fusion protein (R&D Systems) at 10 ug/ml in PBS for 2 h at room temperature. Supernatant was aspirated and 250 ul/well of 3% bovine serum albumin (Sigma) in PBS was added and incubated for 1 h. Antibody was diluted in 1% BSA in PBS and added 100 ul/well after blocking solution was aspirated; antibody solution was incubated for 1 h. Plate was then washed 3 times with 250 ul/well of PBS with 0.5% Tween-20. 100 ul/well of HRP-conjugated anti-FAB antibody (Sigma) diluted 1:1000 in 1% BSA in PBS and incubated for 1 h. Plate was washed as above and 100 ul/well of 3,3′,5,5′-Tetramethylbenzidine (Sigma) was added. When blue color developed, reaction was stopped with 1M H2SO4 and absorption at 450 nm was read. FIG. 7 compares DX IgG with SC-DX IgG, both produced in P. pastoris.

Although the foregoing invention has been described in detail for purposes of clarity of understanding, it will be obvious to one skilled in the art that certain modifications may be practiced within the scope of the present invention set forth in the appended claims. All publications and patent documents cited herein are hereby incorporated by reference in their entirety for all purposes to the same extent as if each were so individually denoted. Unless otherwise apparent from the context each embodiment, feature, aspect, element, or step of the invention can be used in combination with any other embodiment, feature, aspect, element, or step.

1. A method of producing an antibody comprising:
culturing a fungal cell transformed with a nucleic acid encoding a fusion protein comprising in order from N-terminus to C-terminus: (a) a signal sequence, (b) a first immunoglobulin chain comprising a variable region and a constant region, (c) a spacer peptide comprising a proteolytic cleavage site cleavable by a protease which is a separate molecule from the fusion protein, and (d) a second immunoglobulin chain comprising a variable region and a constant region; wherein the first immunoglobulin chain is a light chain and the second immunoglobulin chain is a heavy chain, or vice versa; the fusion protein is free of a second signal sequence between the spacer peptide and the second immunoglobulin chain; and the spacer peptide lacks a self-processing cleavage site;
wherein the fusion protein is expressed, cleaved at the C-terminal end of the signal sequence to remove the signal sequence, and cleaved at the proteolytic site in the spacer peptide by the protease; and
an antibody comprising a pair of intermolecularly associated immunoglobulin heavy and light chains is produced.

2. The method of claim 1, wherein the antibody is a tetrameric antibody comprising two pairs of the intermolecularly associated immunoglobulin heavy and light chains.

3. The method of claim 2, wherein the first immunoglobulin chain is a light chain and the second immunoglobulin chain is a heavy chain.

4. The method of claim 2, wherein the first immunoglobulin chain is a heavy chain and the second immunoglobulin chain is a light chain.

5. The method of claim 2, wherein the light and heavy chains of the fusion protein associate with each other by intramolecular bonding, and two copies of the fusion protein associate with each other by intermolecular bonding of their respective heavy chain constant regions before cleavage at the proteolytic site occurs.

6. The method of claim 2, wherein cleavage at the proteolytic site is followed by intermolecular association of the immunoglobulin heavy and light chains to form the pair of intermolecularly associated heavy and light chains, and intermolecular association between two pairs of the intermolecularly associated heavy and light chains to form the tetrameric antibody.

7. The method of claim 2, wherein the spacer peptide comprises first and second proteolytic cleavage sites cleavable by first and second proteases, both proteases being separate molecules from the fusion protein, wherein the first and second proteolytic cleavage sites are separated by a peptide linker, and cleavage of the proteolytic cleavage sites by the first and second proteases removes the peptide linker from the fusion protein.

8. The method of claim 7, wherein the first and second protease are the same protease.

9. The method of claim 8, wherein the cleavage of the first and second proteolytic sites occurs in the cell.

10. The method of claim 9, wherein the cell secretes the antibody.

11. The method of claim 8, wherein the cell is transformed with a nucleic acid encoding the protease that cleaves the first and second proteolytic sites.

12. The method of claim 11, wherein the nucleic acid encodes a second fusion protein comprising a second signal sequence fused to the protease, wherein the second signal sequence causes uptake of the protease into the endoplasmic reticulum.

13. The method of claim 1, wherein the fusion protein is secreted from the cell without the signal sequence, and the method further comprises treating the secreted fusion protein with the protease, which cleaves the proteolytic site in the spacer peptide.

14. The method of claim 1, further comprising recovering the antibody from the cell or from media in which the cell is cultured.

15. The method of claim 14, further comprising purifying the antibody to essential homogeneity.

16. The method of claim 15, further comprising combining the antibody with a pharmaceutical carrier in a pharmaceutical composition.

17. The method of claim 1, further comprising introducing the nucleic acid encoding the fusion protein into the cell.

18. The method of claim 1, wherein the cell is a filamentous fungus cell.

19. The method of claim 1, wherein the cell is a yeast cell.

20. The method of claim 1, wherein the cell is selected from the group consisting of cells from Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans Aspergillus nidulans, Aspergillus niger, Aspergillus oryzae, Trichoderma reesei, Chrysosporium lucknowense, Fusarium sp., Fusarium gramineum, Fusarium venenatum and Neurospora crassa.

21. The method of claim 10, wherein the proteolytic cleavage sites are Kex2p sites.

22. The method of claim 21, wherein the proteolytic cleavage sites have the amino acid sequence XXKR, where X is any amino acid.

23. The method of claim 21, wherein the proteolytic cleavage sites have the amino acid sequence XXKR, where X is a hydrophobic amino acid selected from the group consisting of met, ala, val, leu, ile, cys, phe, pro, trp, and tyr or a hydrophilic amino acid selected from the group consisting of arg, asn, asp, gln, glu, his, lys, ser, and thr.

24. The method of claim 23, wherein the spacer peptide has an N-terminal proteolytic cleavage site having the amino acid sequence LVKR and a C-terminal proteolytic cleavage site having the amino acid sequence RLVKR.

25. The method of claim 24, wherein the antibody lacks all residues of the spacer peptide.

26. The method of claim 2, wherein the tetrameric antibody has an effector function.

27. The method of claim 26, wherein the effector function is complement fixation or antibody dependent cellular toxicity.

28. The method of claim 1, wherein the immunoglobulin light chain and heavy chain are humanized immunoglobulin light and heavy chains.

29. The method of claim 1, wherein the antibody is produced at a yield of at least 50 mg/liter of culture medium.

30. The method of claim 1, wherein the glycosylation is at least at position Asn297.

31. The method of claim 1, wherein the heavy chain constant region comprises CH1, hinge, CH2, and CH3 regions.

32. The method of claim 31, wherein the heavy chain constant region further comprises as CH4 region.

33. The method of claim 1, further comprising purifying the antibody and incorporating the antibody into a diagnostic kit.

34. The method of claim 1, wherein the fusion protein lacks peptide segments from a host protein between the signal sequence and the first immunoglobulin chain or between the peptide spacer and the second immunoglobulin chain.

35. A nucleic acid encoding a fusion protein comprising in order from N-terminus to C-terminus: (a) a signal sequence, (b) a first immunoglobulin chain comprising a variable region and a constant region, (c) a spacer peptide comprising a proteolytic cleavage site cleavable by a protease which is a separate molecule from the fusion protein, and (d) a second immunoglobulin chain comprising a variable region and a constant region; wherein the first immunoglobulin chain is a light chain and the second immunoglobulin chain is a heavy chain, or vice versa; the fusion protein is free of a second signal sequence between the spacer peptide and the second immunoglobulin chain; and the spacer peptide lacks a self-processing cleavage site.

36. A vector comprising the nucleic acid of claim 34 operably linked to a regulatory sequence.

37. A cell transformed with the nucleic acid of claim 35.

38. An antibody composition comprising a plurality of molecules of an antibody produced by the method of claim 1, wherein each of the plurality has a glycoform, and the predominant glycoform is complex and lacking fucose.

39-41. (canceled)

42. A method of producing an antibody comprising:
culturing a cell transformed with a nucleic acid encoding a fusion protein comprising a signal sequence, an immunoglobulin light chain comprising a variable region and a constant region, a spacer peptide comprising first and second proteolytic cleavage sites cleavable by first and second proteases, which can be the same or different, both of which are separate molecules from the fusion protein, and an immunoglobulin heavy chain comprising a variable region and a constant region, wherein the spacer peptide is free of a self-cleavable proteolytic site, wherein the fusion protein is expressed, cleaved at the C-terminal end of the signal sequence to remove the signal sequence, and cleaved by the first and second proteases at the first and second proteolytic sites in the spacer peptide, and an antibody comprising a pair of intermolecularly associated immunoglobulin heavy and light chains is produced.

43-75. (canceled)
